(19) United States
(12) Patent Application Publication
(10) Pub. No.: US 2003/0188591 A1
(43) Pub. Date: Oct. 9, 2003

(54) Concrete test specimen method and apparatus

(76) Inventor: Gary Workman, Bellwood, IL (US)

Correspondence Address: F. William McLaughlin, Wood, Phillips, Katz, Clark & Mortimer, Suite 3800, 500 West Madison Street, Chicago, IL, 60661, US

(21) Appl. No.: 10/118,399

(22) Filed: Apr. 8, 2002

(51) Int. Cl. G01N 33/00

(52) U.S. Cl. 73/866

(57) ABSTRACT

A concrete test specimen apparatus includes a test cylinder having a cylindrical side wall closed by a bottom wall to define an interior space accessible through a top opening. A rectangular liner in the interior space covers an inside of the cylindrical side wall. When the interior space is filled with a specimen of concrete, the liner is disposed between the specimen and the side wall to enable sliding of the specimen out of the interior space.

### FILED OF THE INVENTION

This invention relates to concrete test specimens and, more particularly, to an improved method and apparatus for stripping concrete test specimens from test cylinders.

### BACKGROUND OF THE INVENTION

In construction projects it is often necessary that the concrete used satisfies certain specifications. In order to ensure that the concrete satisfies these specifications, it is necessary to pass specified tests. Particularly, quality assurance for concrete construction requires samples of concrete be collected and tested according to strict ASTM specifications. These ASTM specifications require concrete to be collected in test cylinders. Normal job site collections specify that a six inch diameter by twelve inch high cylinder be used. Occasionally, a four inch diameter by eight inch high test cylinder is used. Specimens are allowed to set up/harden and are then taken to a test laboratory where the specimens are stripped from the test cylinder, cured for a specified time, then tested for strength.

The test cylinders are conventionally formed of plastic. There are presently two standard procedures for stripping the concrete test specimens from the plastic cylinder. The first method is to slice the wall of the test cylinder with a special stripping tool or blade. This method renders the test cylinder a disposable item. The second method for stripping the concrete test specimens is to punch a hole in the bottom of the plastic test cylinder and introduce compressed air through the hole to blow the specimen from the test cylinder. This enables reuse of the test cylinder by taping over the punched hole.

A problem with the first method is cost. The test cylinder is used once and then disposed of. The cylinder is not recyclable since it may contain concrete residue. Thus, the lab must incur the cost of both the test cylinder and cost of disposal.

The second method can provide reusability of the test cylinder. However, the introduction of compressed air into the bottom of the test cylinder often causes the bottom of the test cylinder to break rather than blowing the specimen from the test cylinder. In addition, the attempt to blow out the specimen often causes the bottom to bow which restricts the side walls of the test cylinder. This causes the edges of the specimen to chip which can impact on the final test results.

The present invention is directed to overcoming one or more of the problems discussed above in a novel and simple manner.

### SUMMARY OF THE INVENTION

In accordance with the invention, there is disclosed an improved method and apparatus for stripping concrete test specimens.

Broadly, the specimen is stripped from a test cylinder by utilizing a liner. The liner is placed into the test cylinder prior to placement of the concrete. After the concrete has set and is ready for stripping, the cylinder is placed upside down so that the specimen slides out of the test cylinder.

Further features and advantages of the invention will readily be apparent from the specification and from the drawings.

### BRIEF DESCRIPTION OF THE DRAWINGS

FIG. 1 is a perspective view of a test cylinder in accordance with the invention;

FIG. 2 is a plan view of a liner used in accordance with the invention;

FIG. 3 is a perspective view of the test cylinder of FIG. 1 with the liner of FIG. 2 inserted therein;

FIG. 4 is a perspective view of the test cylinder of FIG. 3 after filling with concrete;

FIG. 5 is a perspective view of a fixture used to aid in stripping of the test cylinders;

FIG. 6 is a sectional view illustrating stripping of the test cylinder in accordance with the invention; and

FIG. 7 is a sectional view, similar to FIG. 6, illustrating a test specimen sliding out of the test cylinder.

### DETAILED DESCRIPTION OF THE INVENTION

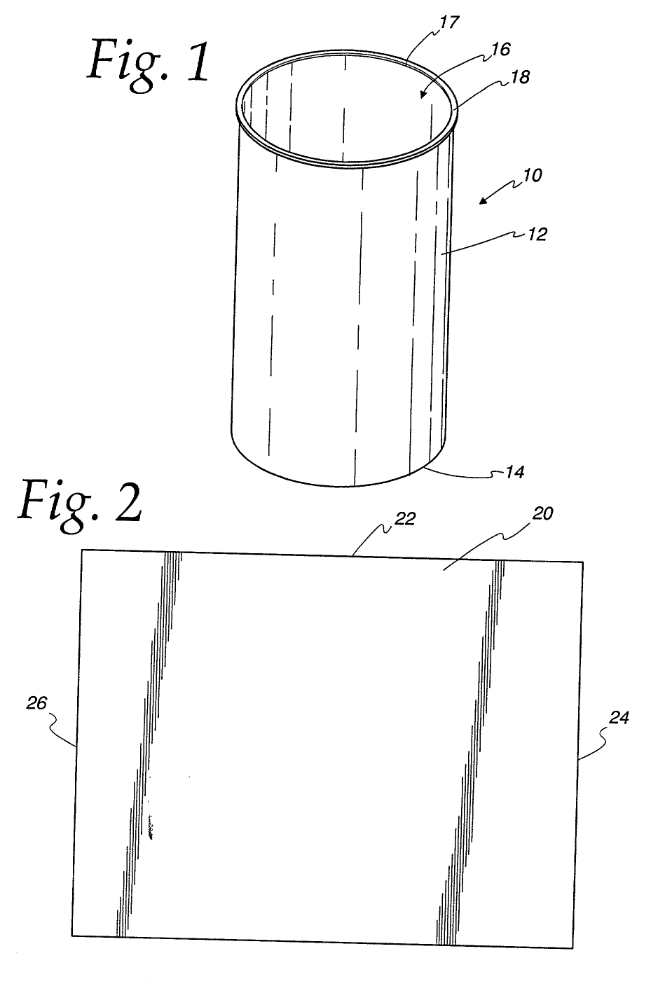

With reference to the drawings, a testing receptacle in the form of a test cylinder 10, see FIG. 1, is used for performing testing accordance to ASTM specifications. The test cylinder 10 comprises a cylindrical side wall 12 closed by a bottom wall 14 to define an interior space 16 accessible through a top opening 17. A flange 18 extends outwardly from a top end of the side wall 12. In the illustrated embodiment of the invention, the test cylinder interior space 16 has a diameter of six inches and a height of twelve inches. As is apparent, other sizes could be used, as necessary or desired, such as, for example, four inches diameter and eight inches height.

In the illustrated embodiment of the invention, the test cylinder 10 is formed of one piece molded plastic. As is apparent, the test cylinder 10 could also be formed of paper or steel, or the like. Moreover, the method described herein could be used in connection with other concrete test apparatus such as slump cones, beam molds, cube molds, etc., in which concrete specimens must be removed. As such, the method and apparatus described herein could be used with different shaped test devices, including conical, rectangular, etc.

FIG. 2 illustrates a perspective view of a liner 20 shown in a flattened state. The liner 20 comprises a sheet 22 having a height corresponding to a height of the side wall 12 and thus space 16 and a length related to the inner circumference of the side wall 12. Particularly, the length must be equal to or larger than the inner circumference of the side wall 12

The sheet 22 can be of various different materials. These materials include wax paper, wax impregnated paper, parchment paper, oil impregnated paper, plastic lined paper, plastic film or sheet, Teflon sheets, or thin sheet metal. Any thin material that prevents a coefficient of adhesion to the cylinder side wall 12 may be used. Advantageously, a material having a smoother surface, i.e., lower coefficient of friction, such as plastic sheet or Teflon sheet, should be used.

The liner 20 can be installed with lateral ends 24 and 26 butted or butted and taped. Likewise, the ends 24 and 26 can be overlapped or overlapped and taped. Additionally, the liner 20 can be freestanding or pushed against the inside of the side wall 12 or taped to the side wall 12. The stiffness of the material and its memory to go back to flat, thus hugging the side wall 12, dictates the preferred method. A material thickness of approximately 3 ml or larger requires no taping and performs best with a slight overlap. ASTM specifications for test cylinder dimensions would preclude anything thicker than about 50 ml.

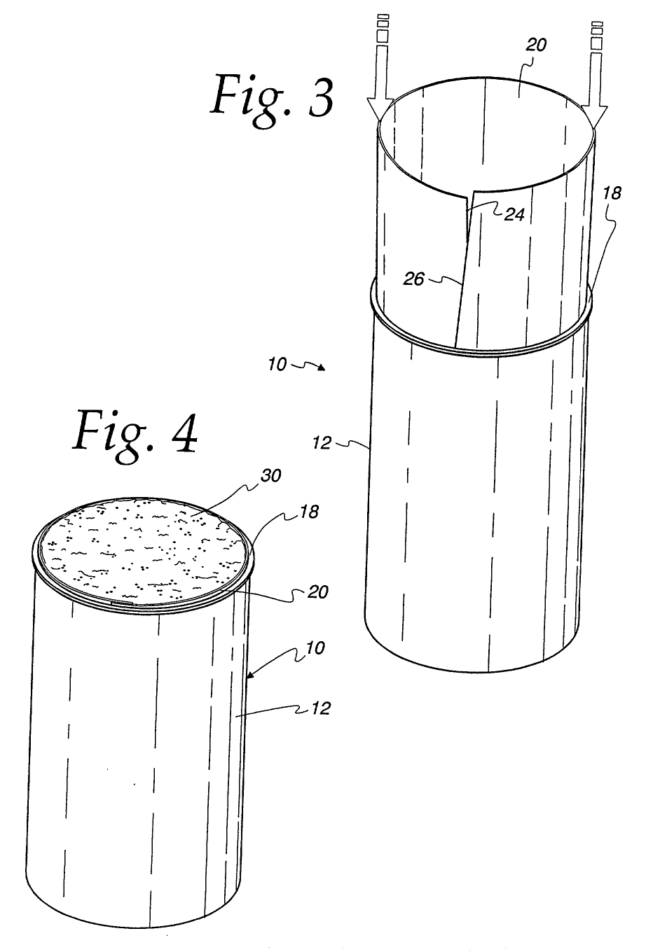

Referring to FIG. 3, the cylinder 10 is illustrated with the liner 20 being inserted in the space 16, as represented by arrows, generally hugging the inside of the side wall 12 with the ends overlapped. Thereafter, the test cylinder 10 is filled with concrete 30 as shown in FIG. 4. After the concrete has set up and hardened, then the specimen 30 can be stripped from the test cylinder 10. The cylinder 10 can be stripped by sliding the test specimen 30 from the interior space 16. The liner 20 enables the specimen 30 to more easily slide out of the interior space 16 and prevents concrete residue from adhering to the side wall 12.

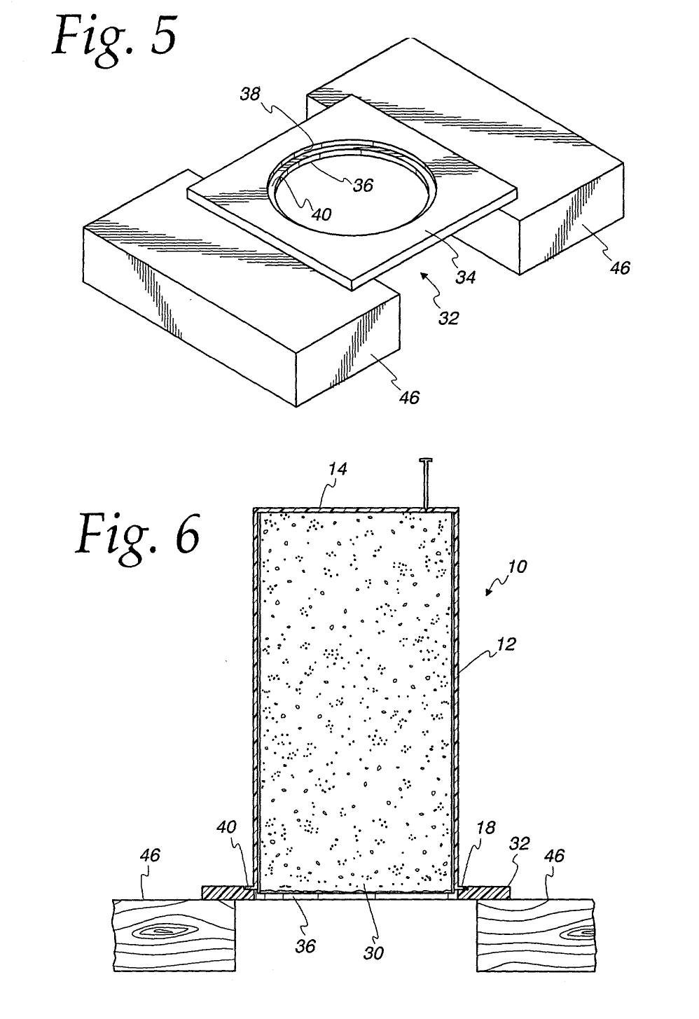

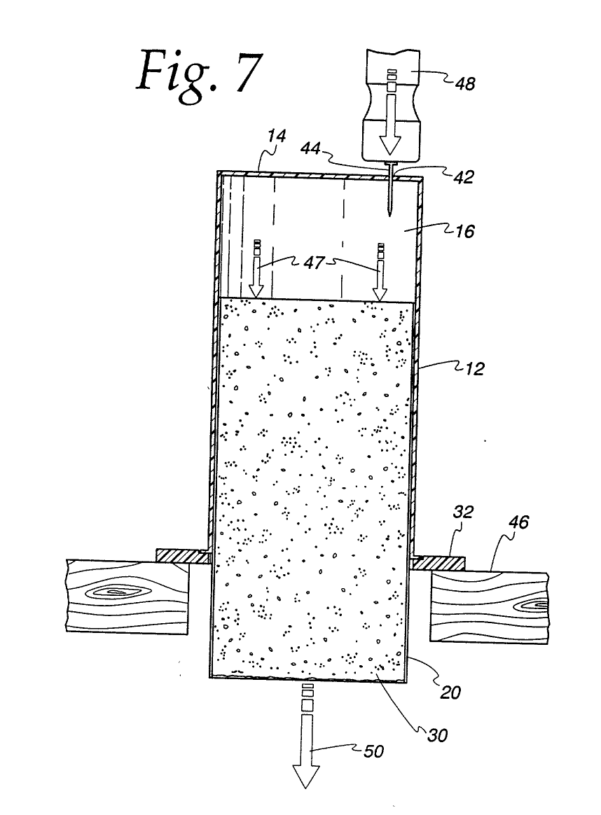

To facilitate stripping of the specimen 30, a stripping fixture 32, see FIG. 5, can be used to enhance labor savings. The stripping fixture 32 allows the test cylinder 10 to remain stationary and the specimen 30 to slide out. The fixture 32 comprises a generally square-shaped plate 34 having a central through opening 36. A counter bore 38 surrounds the through opening 36 to define a shoulder 40. The through opening 36 is sized to be slightly larger than the diameter of the concrete specimen but smaller than the test cylinder flange 18. The diameter of the counter bore 38 is slightly larger than the outside diameter of the flange 18. This permits the test cylinder 10 with the concrete specimen 30 to be placed precisely on the fixture 32 with the flange 18 resting on the shoulder 40, as shown in FIG. 6. Once the test cylinder 10 with specimen 30 is placed upside down onto the fixture 32, gravity begins to allow the specimen 30 to slide out of the test cylinder 10, as shown in FIG. 7, in the direction of the arrows 47. To help, a hole 42 can be punched into the bottom wall 14 with a nail 44, or the like, using a hammer 48 to introduce air and break any vacuum. In the illustrated example of FIG. 7, the liner 20 slides out with the specimen 30 and can then be removed. As is apparent, the liner 20 might also remain in the space 16. By setting up a series of these fixtures, a technician can set several units and allow them to be stripped in a fraction of the time it usually takes using current procedures. The fixture 32 can be resting on blocks 46 to provide room for the specimen 30 to fall through the opening 36, as illustrated by the arrow 50.

As discussed above, the liner 20 can be constructed from a broad range of materials. Moreover, the material could include a spray on or liquid such as silicon spray, form release oil, etc. to assist in removal.

Thus, in accordance with the invention, there is provided an improved system and method for stripping concrete test specimens from test cylinders.

I claim:

1. The method testing concrete comprising:
providing a testing receptacle having a side wall defining an interior space accessible through a top opening;
inserting a liner in the interior space to cover an inside of the side wall;
filling the interior space with a specimen of concrete so that the liner is disposed between the specimen and the side wall; and
subsequent to setup of the specimen, sliding the specimen out of the interior space.

2. The method of claim 1 wherein providing a testing receptacle comprises comprising a test cylinder having a cylindrical side wall.

3. The method of claim 2 wherein the testing receptacle is of plastic construction.

4. The method of claim 1 wherein the liner is of a material selected from a group consisting of wax paper, wax impregnated paper, parchment paper, oil impregnated paper, plastic lined paper, plastic film or sheet, Teflon sheets, or thin sheet metal.

5. The method of claim 1 wherein the liner has a thickness in the range of 3 mil to 50 mil.

6. The method of claim 1 wherein sliding the specimen out of the interior space further comprises introducing an opening into the receptacle to break any vacuum.

7. The method of claim 1 further comprising placing the testing receptacle on a fixture having an opening corresponding to the top opening so the specimen slides out under gravity.

8. A concrete test specimen apparatus comprising:
a testing receptacle having a side wall defining an interior space accessible through a top opening; and
a liner in the interior space to cover an inside of the side wall, wherein when the interior space is filled with a specimen of concrete, the liner is disposed between the specimen and the side wall to enable sliding of the specimen out of the interior space.

9. The concrete test specimen apparatus of claim 8 wherein the side wall is cylindrical and is connected to a bottom wall.

10. The concrete test specimen apparatus of claim 8 wherein the testing receptacle is of plastic construction.

11. The concrete test specimen apparatus of claim 8 wherein the liner is of a material selected from a group consisting of wax paper, wax impregnated paper, parchment paper, oil impregnated paper, plastic lined paper, plastic film or sheet, Teflon sheets, or thin sheet metal.

12. The concrete test specimen apparatus of claim 8 wherein the liner has a thickness in the range of 3 mil to 50 mil.

13. The concrete test specimen apparatus of claim 8 wherein the liner has a smooth surface.

14. The concrete test specimen apparatus of claim 8 further comprising a fixture for supporting the testing receptacle and having an opening corresponding to the top opening so the specimen slides out under gravity.

15. The concrete test specimen apparatus of claim 8 wherein the liner has a thickness of about 3 mil and a length greater than an inner circumference of the side wall.

16. A concrete test specimen apparatus comprising:
a test cylinder having a cylindrical side wall closed by a bottom wall to define an interior space accessible through a top opening; and
a rectangular liner in the interior space to cover an inside of the cylindrical side wall, wherein when the interior space is filled with a specimen of concrete, the liner is disposed between the specimen and the side wall to enable sliding of the specimen out of the interior space.

17. The concrete test specimen apparatus of claim 16 wherein the testing receptacle is of plastic construction.

18. The concrete test specimen apparatus of claim 16 wherein the liner is of a material selected from a group consisting of wax paper, parchment paper, oil impregnated paper, plastic lined paper, plastic film or sheet, Teflon sheets, or thin sheet metal.

19. The concrete test specimen apparatus of claim 16 wherein the liner has a thickness in the range of 3 mil to 50 mil.

20. The concrete test specimen apparatus of claim 16 wherein the liner has a smooth surface.

21. The concrete test specimen apparatus of claim 16 further comprising a fixture for supporting the testing receptacle and having an opening corresponding to the top opening so the specimen slides out under gravity.

22. The concrete test specimen apparatus of claim 16 wherein the liner has a thickness of about 3 mil and a length greater than an inner circumference of the side wall.
